(19) United States
(12) Patent Application Publication
(10) Pub. No.: US 2020/0268314 A1
(43) Pub. Date: Aug. 27, 2020

(54) PAIN INTENSITY LEVEL AND SENSATION PERCEPTION

(71) Applicant: International Business Machines Corporation, Armonk, NY (US)

(72) Inventors: Fang Lu, Billerica, MA (US); Yoonyoung Park, Cambridge, MA (US); Francis Campion, Westwood, MA (US); Uri Kartoun, Cambridge, MA (US)

(21) Appl. No.: 16/285,771

(22) Filed: Feb. 26, 2019

(51) Int. Cl. A61B 5/00; G16H 10/60; G06K 9/62; G06K 9/00; G06N 20/00

(52) U.S. Cl. CPC A61B 5/4827; G16H 10/60; G16H 50/30; G06K 9/00302; G06N 20/00; G06K 9/6267

(57) ABSTRACT

Embodiments provide a computer implemented method of perceiving a pain intensity level and sensation, the method comprising: training, by a processor, a first machine learning model with a plurality of electronic medical records of different patients having a pain; deriving, by the processor, a second machine learning model for a particular patient from the first machine learning model, based on a medical history, all the speech, facial expressions and body language of the particular patient during each clinic visit; receiving, by the second machine learning model, new speech, new facial expressions, and new body language from the particular patient; and generating, by the second machine learning model, a pain intensity level and sensation of the particular patient based on the new speech, new facial expressions, and new body language.

### TECHNICAL FIELD

The present disclosure relates generally to a system, method, and computer program product that can help healthcare professionals and caregivers perceive patients' pain intensity levels.

### BACKGROUND

It is a challenge for a healthcare professional to understand a patient's pain intensity level or how exactly the patient feels, because each person experiences pain differently, each person may have different tolerance to pain, and it is difficult for the patient to describe the pain intensity level in words.

Further, physicians are increasingly reluctant to prescribe opioids as painkillers, in fear of government scrutiny or malpractice lawsuits. There is a critical need for the healthcare professionals and caregivers to perceive more precisely how the patient feels, so that an appropriate pain management decision can be provided.

### SUMMARY

Embodiments provide a computer implemented method in a data processing system comprising a processor and a memory comprising instructions, which are executed by the processor to cause the processor to implement the method of perceiving a pain intensity level of a patient, the method comprising: training, by the processor, a first machine learning model with a plurality of electronic medical records of different patients having a pain; deriving, by the processor, a second machine learning model for a particular patient from the first machine learning model, based on a medical history and a patient attribute of the particular patient during each clinic visit; receiving, by the second machine learning model, a new patient attribute from the particular patient collected during the current clinic visit; and generating, by the second machine learning model, a pain intensity level of the particular patient based on the new patient attribute.

Embodiments further provide a computer implemented method, further comprising: providing, by the second machine learning model, the pain intensity level of the particular patient to a simulation device; simulating, by the simulation device, the pain intensity level of the particular patient; and providing, by the simulation device, the simulated pain intensity level of the particular patient to a physician.

Embodiments further provide a computer implemented method, further comprising: identifying, by the first machine learning model, a patient condition of the particular patient based on the new patient attribute, wherein the new patient attribute comprises one or more of: new speech, new facial expressions, and new body language; and generating, by the second machine learning model, a sensation of the patient condition of the particular patient.

Embodiments further provide a computer implemented method, further comprising: providing, by the second machine learning model, the pain intensity level and the sensation of the particular patient to a simulation device; simulating, by the simulation device, the pain intensity level and the sensation of the particular patient; and providing, by the simulation device, the simulated pain intensity level and the simulated sensation of the particular patient to a physician.

Embodiments further provide a computer implemented method, wherein the simulation device comprises one or more of: an augmented reality device, a virtual reality device, a mixed reality device, and an extended reality device.

Embodiments further provide a computer implemented method, further comprising: training the first machine learning model through linear regression.

Embodiments further provide a computer implemented method, wherein the patient attribute of the particular patient during each clinic visit is included in an electronic medical record of the particular patient, wherein the patient attribute is described by the physician in the electronic medical record in an electronic text format, wherein the patient attribute comprises one or more of: speech, facial expressions and body language.

In another illustrative embodiment, a computer program product comprising a computer usable or readable medium having a computer readable program is provided. The computer readable program, when executed on a processor, causes the processor to perform various ones of, and combinations of, the operations outlined above with regard to the method illustrative embodiment.

In yet another illustrative embodiment, a system is provided. The system may comprise a processor configured to perform various ones of, and combinations of, the operations outlined above with regard to the method illustrative embodiments.

Additional features and advantages of this disclosure will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawing embodiments that are presently preferred, it being understood, however, that the invention is not limited to the specific instrumentalities disclosed. Included in the drawings are the following Figures:

FIG. 1 depicts a block diagram of an example pain intensity level perception system 100, according to embodiments herein;

FIG. 2 is an example flowchart illustrating a method 200 of perceiving a pain intensity level of a particular patient, according to embodiments herein;

FIG. 3 is another example flowchart illustrating a method 300 of perceiving a pain intensity level of a particular patient, according to embodiments herein; and

FIG. 4 is a block diagram of an example data processing system 400 in which aspects of the illustrative embodiments may be implemented.

### DETAILED DESCRIPTION OF EXEMPLARY EMBODIMENTS

Embodiments of the present invention may be a system, a method, and/or a computer program product. The computer program product may include a computer readable storage medium (or media) having computer readable program instructions thereon for causing a processor to carry out aspects of the present invention.

In an embodiment, a system, method, and computer program product for healthcare professionals to understand patients' pain intensity levels, are disclosed. Specifically, a role exchange platform is established for the healthcare professionals to understand how the patients feel by utilizing a simulation device, such as an AR (Augmented Reality), VR (Virtual Reality), MR (Mixed Reality) or XR (Extended Reality) based device, and the like. The system, method, and computer program product simulates the patient's feeling based on a plurality of factors, such as patient's speech, facial expressions and body language, and the healthcare professionals, e.g., a physician, can feel the simulated pain or/and sensation (e.g., hot or cold) through a pair of AR, VR, MR, or XR glasses. The physician can then decide whether a painkiller should be prescribed to this patient or not.

In an embodiment, a machine learning model is built based on a huge number of electronic medical records (EMR) of different patients, who have various pain and sensation symptoms. For example, electronic medical records used for training the machine learning model can include records of pain such as stomachache, headache, toothache, leg ache, etc., and sensations such as chilling, cold, medium warm, warm, medium hot, hot, etc. The machine learning model can be continuously trained with new EMR information from different patients. In an embodiment, the machine learning model can be trained through linear regression. The machine learning model can be either supervised machine learning model or unsupervised machine learning model.

In a further embodiment, a personalized machine learning model for a particular patient is derived from the aforementioned machine learning model, based on the past medical history, speech, facial expressions and body language of the particular patient. Each patient has different pain tolerance, and thus the personalized machine learning model is derived considering each patient's own features, such as a personal medical history, personal speech, personal facial expressions and personal body language. The personalized machine learning model can be continuously trained using new EMR information of the particular patient. For example, each time the particular patient visits a clinic, the updated EMR information will be used to train the personalized machine learning model.

In an embodiment, the patient's current behaviors, e.g., the current speech, facial expressions and body language, are input into the personalized machine learning model. The trained personalized machine learning model can output a pain intensity level (e.g., pain scale represented as 0, 1, 2 . . . , 10).

In an embodiment, the trained personalized machine learning model can also output a sensation related to a sickness of the particular patient. Specifically, the machine learning model, trained by a huge amount of EMR information, can decide which sickness the particular patient is subject to, based on the current speech, facial expressions and body language of the particular patient. The sensation, e.g., chilling, sweeting, warm, medium warm, hot, medium hot, etc., corresponding to the identified sickness is also output either from the machine learning model or the personalized machine learning model.

In an embodiment, the pain intensity level and the sensation are output to a simulation device, e.g., VR, AR, MR, or XR device (represented as a pair of VR, AR, MR, or XR glasses). A plurality of commonly known symptoms, such as pain, dizziness, cold sensation, hot sensation, etc., can be simulated through conventional VR, AR, MR, or XR technologies. The physician can then wear the VR, AR, MR, or XR enabled glasses, and experience the particular patient's pain intensity level and sensation. In an embodiment, an electronic or paper questionnaire can be filled out by the particular patient, as a secondary validation regarding the particular patient's pain intensity level or discomfort level.

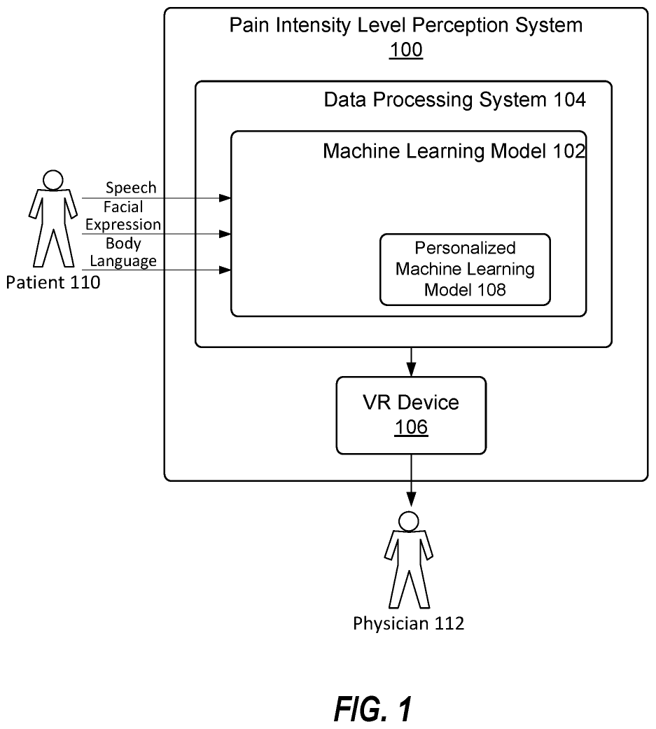

FIG. 1 depicts a block diagram of an example pain intensity level perception system 100, according to embodiments herein. The pain intensity level perception system 100 includes the machine learning model 102 implemented on the data processing system 104, and the VR device 106. The data processing system 104 is wired or wirelessly connected to the VR device 106. In one embodiment, the data processing system 104 is connected to the VR device 106 via a short-range radio network, e.g., Bluetooth®, WiFi®, Zigbee, etc.

As shown in FIG. 1, the data processing system 104 is an example of a computer, such as a server or client, in which computer usable code or instructions implementing the process for illustrative embodiments of the present invention are located. In one embodiment, the data processing system 104 represents a computing device, which implements an operating system. In some embodiments, the operating system can be Windows, Unix system or Unix-like operating systems, such as AIX, A/UX, HP-UX, IRIX, Linux, Minix, Ultrix, Xenix, Xinu, XNU, and the like.

In the depicted example, the machine learning model 102 is trained by medical information of a large number of patients. The electronic medical records of these patients are used to train the machine learning model 102, and the electronic medical records are related to various patient conditions—e.g., an injury, illness, sickness, etc., which may result in pain. The machine learning model 102 can output a pain intensity level, and a sensation corresponding to an associated sickness, based on an input from the patient 110. The input from the patient 110 can include speech (e.g., a conversation with a physician), facial expressions (i.e., one or more motions or positions of the muscles beneath the skin of the face, such as smile, frown, etc.) and body language (e.g., body postures, gestures, eye movements, etc.) and the like.

In an embodiment, the personalized machine learning model 108 for the patient 110 is derived from the machine learning model 102, based on the past medical history recorded in, for example, the EMR of the patient 110, and the past speech, facial expressions and body language of the patient 110 in, for example, previous clinic visits. The personalized machine learning model 108 takes into account the personal medical information of the patient 110, and thus the generated pain intensity level and sensation of the patient 110 will be more accurate. The current speech, facial expressions and body language of the patient 110 in the current clinic visit are input to the trained machine learning model 102 (including the trained personalized machine learning model 108), then a pain intensity level and a sensation of the patient 110 are generated by the machine learning model 102 (including the trained personalized machine learning model 108).

In an embodiment, the pain intensity level and the sensation of the patient 110 are sent to the simulation device, for example, the VR device 106 (e.g., a pair of VR enabled glasses). The VR device 106 can simulate the pain intensity level and the sensation of the patient 110 through conventional technologies. The physician 112 can wear the VR device 106, and feel the simulated pain intensity level and the sensation of the patient 110. The physician 112 can then diagnose and decide further treatment (e.g., prescription) based on the experience of the simulated pain intensity level and the sensation of the patient 110 that the physician 112 is experiencing through the VR device 106.

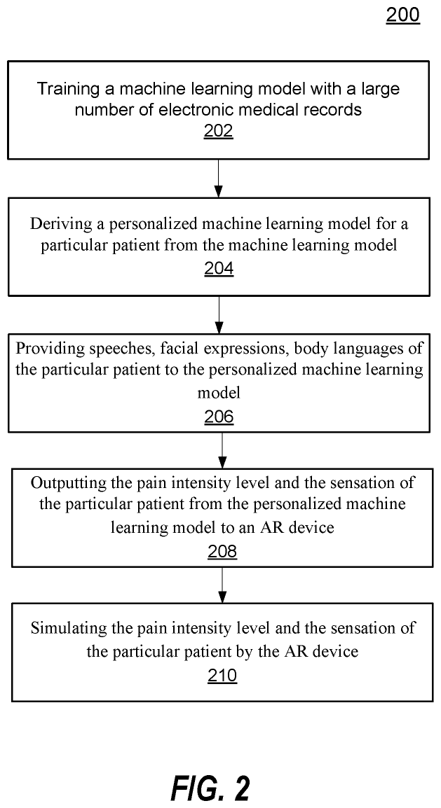

FIG. 2 is an example flowchart illustrating a method 200 of perceiving a pain intensity level of a particular patient, according to embodiments herein. At step 202, the machine learning model 102 is trained with a large number of electronic medical records of different patients. Each patient has his/her electronic medical record describing symptoms of his/her sickness. In an embodiment, the electronic medical record can also include a description about the speech, facial expressions and body language during each clinic visit. In one embodiment, the description of the speech, facial expressions and body language can be provided by a physician or other medical staff. For example, the physician or medical staff can write down the specific patient attributes of the patient 110, such as speech, facial expressions, and body language, from observations. In another embodiment, speech of the patient 110 can be converted into texts through speech recognition. In another embodiment, the facial expressions and body language of the patient 110 may be converted into texts from pictures or videos.

At step 204, the personalized machine learning model 108 for the patient 110 is derived from the machine learning model 102. Specifically, a medical history included in the EMR of the patient 110, and all the patient attributes (e.g., speech, facial expressions and body language) of the patient 110 described or collected by a physician, or a data collection device, during each clinic visit, are used to derive the personalized machine learning model 108 from the machine learning model 102. Because the personalized machine learning model 108 is customized for the patient 110, the pain intensity level and the sensation of the patient 110 output from the personalized machine learning model 108 will be more precise for that particular patient.

At step 206, the speech, facial expressions, body language of the patient 110 during the current clinic visit are input into the personalized machine learning model 108. In an embodiment, the patient attributes (e.g., speech, facial expressions, body language) are described by a physician, or a data collection device, in a text format, and included in the EMR of the patient 110.

At step 208, the pain intensity level and the sensation of the patient 110 are output from the personalized machine learning model 108 to an AR device. The AR device is wired or wirelessly connected to a computing device on which the personalized machine learning model 108 is implemented.

At step 210, the AR device simulates the pain intensity level and the sensation of the patient 110 and conveys the simulation to the physician. Accordingly, the physician can experience the pain intensity level and the sensation of the patient 110, and diagnosis and further treatment (e.g., a prescription) can be determined based on the simulated results.

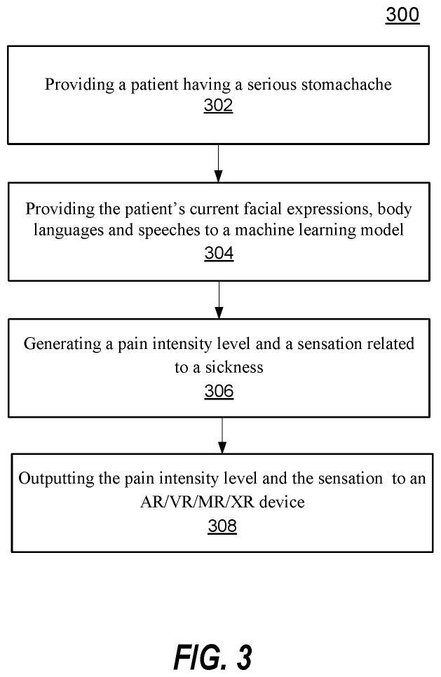

FIG. 3 is another example flowchart illustrating a method 300 of perceiving a pain intensity level of a particular patient, according to embodiments herein. At step 302, a patient has a condition of a serious stomachache. It is so painful, and he almost curls up in a ball, mildly panicking. He is not sure whether the stomachache is due to food poisoning, or appendicitis, etc.

At step 304, the patient's current facial expressions (e.g., frown), body language (e.g., curling up in a ball) and speech (e.g., “cannot tolerate the pain”) are input to a computing device implementing the trained machine learning model 102.

At step 306, the trained machine learning model 102 can determine a pain intensity level based on the current facial expressions, body language and speech of the patient. The trained machine learning model 102 can further suggest which sickness the patient may be subject to, and identify a sensation related to the sickness.

At step 308, the pain intensity level and the sensation can be sent to a simulation device (e.g., AR/VR/MR/XR device) for simulation. A physician can experience the simulated pain and sensation, which facilitate identification of the cause of the patient's pain. This aids the physician in determining a treatment plan.

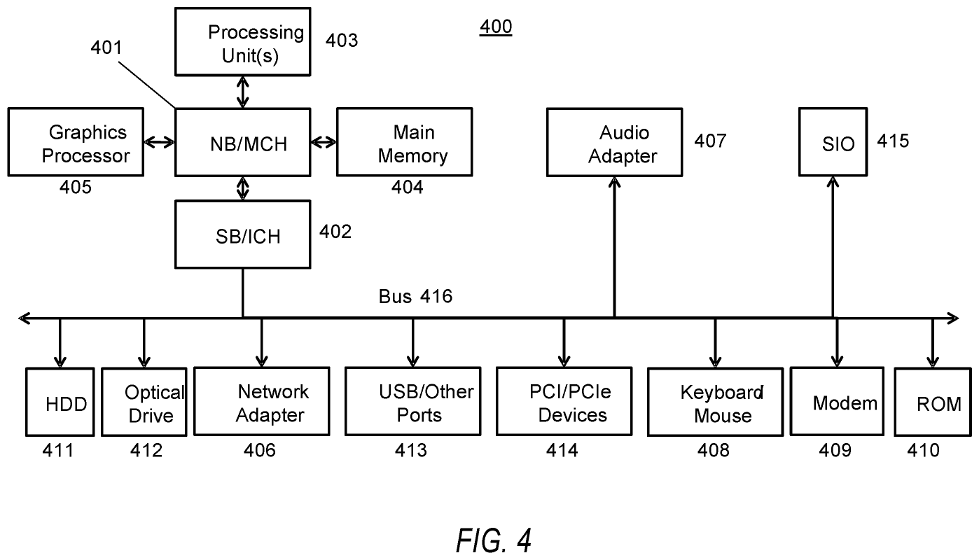

FIG. 4 is a block diagram of an example data processing system 400 in which aspects of the illustrative embodiments may be implemented. The data processing system 400 is an example of a computer, such as a server or client, in which computer usable code or instructions implementing the process for illustrative embodiments of the present invention are located. In one embodiment, FIG. 4 may represent a server computing device.

In the depicted example, data processing system 400 may employ a hub architecture including a north bridge and memory controller hub (NB/MCH) 401 and south bridge and input/output (I/O) controller hub (SB/ICH) 402. Processing unit 403, main memory 404, and graphics processor 405 may be connected to the NB/MCH 401. Graphics processor 405 may be connected to the NB/MCH 401 through an accelerated graphics port (AGP) (not shown in FIG. 4).

In the depicted example, the network adapter 406 connects to the SB/ICH 402. The audio adapter 407, keyboard and mouse adapter 408, modem 409, read only memory (ROM) 410, hard disk drive (HDD) 411, optical drive (CD or DVD) 412, universal serial bus (USB) ports and other communication ports 413, and the PCI/PCIe devices 414 may connect to the SB/ICH 402 through bus system 416. PCI/PCIe devices 414 may include Ethernet adapters, add-in cards, and PC cards for notebook computers. ROM 410 may be, for example, a flash basic input/output system (BIOS). The HDD 411 and optical drive 412 may use an integrated drive electronics (IDE) or serial advanced technology attachment (SATA) interface. The super I/O (SIO) device 415 may be connected to the SB/ICH 402.

An operating system may run on processing unit 403. The operating system could coordinate and provide control of various components within the data processing system 400. As a client, the operating system may be a commercially available operating system. An object-oriented programming system, such as the Java™ programming system, may run in conjunction with the operating system and provide calls to the operating system from the object-oriented programs or applications executing on data processing system 400. As a server, the data processing system 400 may be an IBM® eServer™ System p® running the Advanced Interactive Executive operating system or the Linux operating system. The data processing system 400 may be a symmetric multiprocessor (SMP) system that may include a plurality of processors in the processing unit 403. Alternatively, a single processor system may be employed.

Instructions for the operating system, the object-oriented programming system, and applications or programs are located on storage devices, such as the HDD 411, and are loaded into the main memory 404 for execution by the processing unit 403. The processes for embodiments of the generation system may be performed by the processing unit 403 using computer usable program code, which may be located in a memory such as, for example, main memory 404, ROM 410, or in one or more peripheral devices.

A bus system 416 may be comprised of one or more busses. The bus system 416 may be implemented using any type of communication fabric or architecture that may provide for a transfer of data between different components or devices attached to the fabric or architecture. A communication unit such as the modem 409 or network adapter 406 may include one or more devices that may be used to transmit and receive data.

Those of ordinary skill in the art will appreciate that the hardware depicted in FIG. 4 may vary depending on the implementation. Other internal hardware or peripheral devices, such as flash memory, equivalent non-volatile memory, or optical disk drives may be used in addition to or in place of the hardware depicted. Moreover, the data processing system 400 may take the form of a number of different data processing systems, including but not limited to, client computing devices, server computing devices, tablet computers, laptop computers, telephone or other communication devices, personal digital assistants, and the like. Essentially, the data processing system 400 may be any known or later developed data processing system without architectural limitation.

The computer readable storage medium may be a tangible device that may retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein may be downloaded to respective computing/processing devices from a computer readable storage medium or to an external computer or external storage device via a network, for example, the Internet, a local area network (LAN), a wide area network (WAN), and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, may be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that may direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts or carry out combinations of special purpose hardware and computer instructions.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one may also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art, in view of the present description, that there are many other alternative implementations for these various elements that may be utilized in addition to, or in replacement of, the examples provided herein without departing from the spirit and scope of the present invention.

The system and processes of the figures are not exclusive. Other systems, processes, and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers and processes may be implemented using hardware components, software components, and/or combinations thereof. No claim element herein is to be construed under the provisions of 35 U.S.C. 112 (f) unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A computer implemented method in a data processing system comprising a processor and a memory comprising instructions, which are executed by the processor to cause the processor to implement the method of perceiving a pain intensity level of a patient, the method comprising:
training, by the processor, a first machine learning model with a plurality of electronic medical records of different patients having a pain;
deriving, by the processor, a second machine learning model for a particular patient from the first machine learning model, based on a medical history and a patient attribute of the particular patient during each clinic visit;
receiving, by the second machine learning model, a new patient attribute from the particular patient collected during the current clinic visit; and
generating, by the second machine learning model, a pain intensity level of the particular patient based on the new patient attribute.

2. The method as recited in claim 1, further comprising:
providing, by the second machine learning model, the pain intensity level of the particular patient to a simulation device;
simulating, by the simulation device, the pain intensity level of the particular patient; and
providing, by the simulation device, the simulated pain intensity level of the particular patient to a physician.

3. The method as recited in claim 1, further comprising:
identifying, by the first machine learning model, a patient condition of the particular patient based on the new patient attribute, wherein the new patient attribute comprises one or more of: new speech, new facial expressions, and new body language; and
generating, by the second machine learning model, a sensation of the patient condition of the particular patient.

4. The method as recited in claim 3, further comprising:
providing, by the second machine learning model, the pain intensity level and the sensation of the particular patient to a simulation device;
simulating, by the simulation device, the pain intensity level and the sensation of the particular patient; and
providing, by the simulation device, the simulated pain intensity level and the simulated sensation of the particular patient to a physician.

5. The method as recited in claim 4, wherein the simulation device comprises one or more of: an augmented reality device, a virtual reality device, a mixed reality device, and an extended reality device.

6. The method as recited in claim 4, further comprising: training the first machine learning model through linear regression.

7. The method as recited in claim 2, wherein the patient attribute of the particular patient during each clinic visit is included in an electronic medical record of the particular patient, wherein the patient attribute is described by the physician in the electronic medical record in an electronic text format, wherein the patient attribute comprises one or more of: speech, facial expressions and body language.

8. A computer program product for perceiving a pain intensity level of a patient, the computer program product comprising a computer readable storage medium having program instructions embodied therewith, the program instructions executable by a processor to cause the processor to:
train a first machine learning model with a plurality of electronic medical records of different patients having a pain;
derive a second machine learning model for a particular patient from the first machine learning model, based on a medical history and a patient attribute of the particular patient during each clinic visit;
receive, by the second machine learning model, a new patient attribute from the particular patient; and
generate, by the second machine learning model, a pain intensity level of the particular patient based on the new patient attribute.

9. The computer program product as recited in claim 8, wherein the processor is further caused to:
provide, by the second machine learning model, the pain intensity level of the particular patient to a simulation device;
simulate, by the simulation device, the pain intensity level of the particular patient; and
provide, by the simulation device, the simulated pain intensity level of the particular patient to a physician.

10. The computer program product as recited in claim 8, wherein the processor is further caused to:
identify, by the first machine learning model, a patient condition of the particular patient based on the new patient attribute, wherein the new patient attribute comprises one or more of: new speech, new facial expressions, and new body language; and
generate, by the second machine learning model, a sensation of the patient condition of the particular patient.

11. The computer program product as recited in claim 10, wherein the processor is further caused to:
provide, by the second machine learning model, the pain intensity level and the sensation of the particular patient to a simulation device;
simulate, by the simulation device, the pain intensity level and the sensation of the particular patient; and
provide, by the simulation device, the simulated pain intensity level and the simulated sensation of the particular patient to a physician.

12. The computer program product as recited in claim 11, wherein the simulation device comprises one or more of: an augmented reality device, a virtual reality device, a mixed reality device, and an extended reality device.

13. The computer program product as recited in claim 10, wherein the processor is further caused to:
train the first machine learning model through linear regression.

14. The computer program product as recited in claim 11, wherein the patient attribute of the particular patient during each clinic visit is included in an electronic medical record of the particular patient, wherein the patient attribute is described by the physician in the electronic medical record in an electronic text format, wherein the patient attribute comprises one or more of: speech, facial expressions and body language.

15. A system for perceiving a pain intensity level of a patient, comprising:
a simulation device, configured to simulate the pain intensity level of a particular patient; and
a processor configured to:
train a first machine learning model with a plurality of electronic medical records of different patients having a pain;
derive a second machine learning model for the particular patient from the first machine learning model, based on a medical history and a patient attribute of the particular patient during each clinic visit;
receive, by the second machine learning model, a new patient attribute from the particular patient;
generate, by the second machine learning model, a pain intensity level of the particular patient based on the new patient attribute;
provide, by the second machine learning model, the pain intensity level of the particular patient to the simulation device;
simulate, by the simulation device, the pain intensity level of the particular patient; and
provide, by the simulation device, the simulated pain intensity level of the particular patient to a physician.

16. The system as recited in claim 15, wherein the processor is further configured to:
identify, by the first machine learning model, a patient condition of the particular patient based on the new patient attribute, wherein the new patient attribute comprises one or more of: new speech, new facial expressions, and new body language; and
generate, by the second machine learning model, a sensation of the patient condition of the particular patient.

17. The system as recited in claim 16, wherein the processor is further configured to:
provide, by the second machine learning model, the sensation of the patient condition of the particular patient to the simulation device;
simulate, by the simulation device, the sensation of the patient condition of the particular patient; and
provide, by the simulation device, the simulated pain intensity level and the simulated sensation of the patient condition of the particular patient to the physician.

18. The system as recited in claim 17, wherein the simulation device comprises one or more of: an augmented reality device, a virtual reality device, a mixed reality device, and an extended reality device.

19. The system as recited in claim 17, wherein the processor is further caused to:
provide an electronic questionnaire to the particular patient, wherein the electronic questionnaire includes a plurality of questions regarding the pain intensity level.

20. The system as recited in claim 17, wherein the patient attribute of the particular patient during each clinic visit is included in an electronic medical record of the particular patient, wherein the patient attribute is described by the physician in the electronic medical record in an electronic text format, wherein the patient attribute comprises one or more of: speech, facial expressions and body language.
